(19) United States
(12) Patent Application Publication
(10) Pub. No.: US 2013/0216574 A1
(43) Pub. Date: Aug. 22, 2013

(54) KIT PROVIDING MULTIPLE UNMET THERAPEUTIC EFFECTS

(71) Applicant: James Liu, Downingtown, PA (US)

(72) Inventor: James Liu, Downingtown, PA (US)

(21) Appl. No.: 13/400,122

(22) Filed: Feb. 20, 2012

(51) Int. Cl. A61K 39/145; A61K 33/04; A61K 33/28; A61K 36/28; A61K 36/61; A61K 33/42; A61K 33/44; A61K 33/24; A61P 29/00; A61P 31/00; A61P 37/08; A61P 11/00; A61P 11/06; A61P 11/02; A61P 37/04; A61P 31/16; A61K 31/167; A61K 36/714

(52) U.S. Cl. 424/209.1; 424/725; 424/705; 424/702; 424/644; 424/764; 424/737; 424/742; 424/604; 424/125; 424/601; 424/651; 514/629

(57) ABSTRACT

The present invention discloses a unique kit for providing a combinational therapeutic product comprising a medical device and a pharmaceutical composition to treat mammal diseases. The medical device is to meet these unmet needs of cleansing out these harmful substances, such as viruses, multiple drug resistant bacteria, fungi, pollen, dusts, or excessive mucus. The pharmaceutical composition adds the required functions of anti-inflammatory, anti-allergy, anti-cancer, promoting membrane healing or immunomodulating, to have the added effect of the medical device. The method of using the combinational product is also disclosed. The new product can be used to prevent and treat a variety of diseases, such as common cold, drug resistant influenza, sinusitis, post nasal drip, virus-triggered asthma, or chronic obstructive pulmonary disease. In addition, the new kit and the process of use are safe and cost effective.

### TECHNICAL FIELD OF THE INVENTION

The present invention relates to a kit for a combinational use of a medical device and pharmaceutical compositions to treat mammal diseases. The medical device plays a key role in meeting a number of the unmet medical needs in cleansing out these harmful substances, such as viruses, multiple drug resistant bacteria, fungi, pollen, dusts, or excessive mucus. The pharmaceutical composition adds the required functions of anti-inflammatory, anti-allergy, anti-cancer, promoting membrane healing or immunomodulating, to compensate what is lack of the medical device. The combinational use of the device and the pharmaceutical preparation can treat a variety of diseases, such as common cold, drug resistant influenza, sinusitis, post nasal drip, virus-triggered asthma, snore, chronic obstructive pulmonary disease (COPD). As part of the invention, the pharmaceutical preparation can be in the solid, liquid or vapor form with a unique formulation for treating a specific disorder. The new process is not only effective in treating both microbial infectious and inflammation, but also does not cause antibiotic resistance. In addition, the new kit and the process of using it is very safe and cost effective.

### BACKGROUND OF THE INVENTION

The common cold is a viral infection of the nose. The common cold is the most common disease worldwide. There is no vaccine to prevent colds, neither any drug to cure colds. Any one of these two hundred cold viruses can cause the common cold. However, the viruses must replicate to have enough infectious particles to cause a symptomatic disease. To win the battle against the cold virus, any effective therapy must reduce the virus number in the disease area—the nose.

When cold viruses invade the epithelial cells in the nasal cavity, they grow to make more. The body's immune system sends in as many disease fighters as possible and releases many kinds of molecules to stop the viral growth. The fighting results in an inflammation. Inflammation, including edema (swelling), redness, and the extra secretions coming out of the nose are the reasons anybody gets a sore throat, nasal congestion, sneeze, cough, and other symptoms. Therefore, the symptoms are the results of a simple battle between the virus and the body's immune system through inflammation. Any effective therapy for the common cold must have the significant anti-inflammation effect, in addition to anti-viral effect.

Many other respiratory tract infections have the similar pathogenic process, such as influenza. Although there are vaccines and drugs for managing influenza, these vaccines and drugs have their limitations due to virus mutation and flu drug resistance. A new combinational therapy having both anti-viral and anti-inflammation effects must be developed for meeting the challenge of the post-flu drug era.

Respiratory syncytial viruses A and B, SARS virus, adenovirus, parainfluenza viruses and the like are common causes for respiratory tract infections in humans. Currently, the medical community has no effective drugs or vaccines or other means to treat or prevent these viral infections, before or even after identification of the viral pathogens. Human adults and school children are constantly exposed to these infectious agents at work and/or at school, and may also be carriers of these infectious agents to and from their home. Any effective method for preventing and treating these vital diseases must have the significant anti-virus and anti-inflammation effect.

The human body is the natural host for many kinds of pathogenic microorganisms. Sinus and nasopharyngeal mucous is one of the prominent places of viral shedding. The nasopharyngeal shedding of these pathogens is the major cause of person-to-person transmission. One skilled in the art will appreciate that those communicable pathogenic microorganisms present in the nasal, sinus and nasopharyngeal cavities will be decreased in quantity after the cavities have been cleansed out after performing an effective nasal irrigation. After sinonasal cleaning, these infectious agents will be less likely to spread horizontally to non-carriers and/or vertically to the lower respiratory tract of the carrier. However, to treat the patient more effectively, the anti-inflammation agent should be utilized.

Numerous medical studies have demonstrated that upper respiratory tract infections with cold viruses trigger asthma attacks. These airborne allergens are also the triggers for inducing asthma attack. For managing asthma, it is the best to prevent its occurrence, not wait for its attack then to start the treatment. The current asthma drugs are for anti-inflammation and for reducing the symptom, but they do not have the function of cleansing out these triggers. Any effective method for preventing the asthma attack must have the significant capacity of eliminating the asthma triggers.

A recent medical study reports that antibiotics have no effects in treating acute sinusitis. The reason is that most acute sinusitis is caused by viruses. These antibiotics have no activity in suppressing viral infection. Any effective therapy for treating sinusitis must have the significant anti-viral and anti-inflammation effect.

Respiratory viruses have been implicated as triggers that worsen chronic obstructive pulmonary disease (COPD). Human rhinovirus is the most common player in increasing the severity of COPD symptoms, lung function changes, and inflammation in exacerbations of COPD. The changes in lower airway inflammation and lung function are more pronounced in proven rhinoviral and putative viral infections. Patients with severe COPD demonstrated greater rises in airway and systemic inflammation than those with milder forms of the disease. This suggests that the mixed nature of exacerbation severity is dependent not only on the nature of infective triggers but also on the inflammation status. Therefore, the patients with COPD need to be treated with a combinational therapy with the significant anti-viral and anti-inflammation effects.

Currently, theses modern antibiotics gradually become less effective due to multiple drug resistance developed by many microbes. A pharmaceutical formulation with a single chemical drug is hard to achieve therapeutic efficacy for a variety of conditions, such as chronic allergic fungal rhinosinusitis. Therefore, an alternative method and process must be developed to treat human diseases. The present invention is a combinational use of a medical device to eliminate viruses and with a pharmaceutical composition containing a number of active components to have a multiple effect in treating human diseases.

Recently, there are a number of commercially available nasal irrigation products. Although these nasal irrigation products are believed to be beneficial, they have not well been clinically studied for treating common cold. Moreover, most nasal irrigation systems use saline to physically remove these pathogens. Saline alone is hardly to have a strong anti-inflammation or anti-allergy efficacy. To improve the overall therapeutic effect for any viral diseases by performing nasal irrigation, additional anti-inflammation efficacy should be added by combinational use of a pharmaceutical preparation.

Previously, I invented a nasal-nasopharyngeal-cleaning (NNC) system used to remove harmful substances from a human's nasal and nasopharyngeal cavities (Liu, Nasal-nasopharyngeal-cleaning system, U.S. Pat. Nos. 6,238,377 May 29, 2001; and 6,736,792, May 18, 2004). This system won the only “Best New Product” Award during the 2010 Efficient Collaborative Retail Marketing (ECRM) Conference for Cough & Cold and Allergy. This system has been clinically shown to remove cold-flu viruses and caused an average of 4.5 shorter duration of the common cold when used three times a day. The cold-flu viruses were removed out effectively from the nasal secretion by these patients participated in the clinical trial (Ao et al. JIDI 2011). However, this award-winning system did not teach a combinational use with a therapeutic anti-inflammation drug, nor the self-healing component. The current invention is to have a unique kit for performing an effective nasal cleanse, providing a functional liquid to be delivered into sinonasal cavities to gain an overall therapeutic effect, and having the self-healing capacity.

### DETAILED DESCRIPTION OF THE DRAWINGS

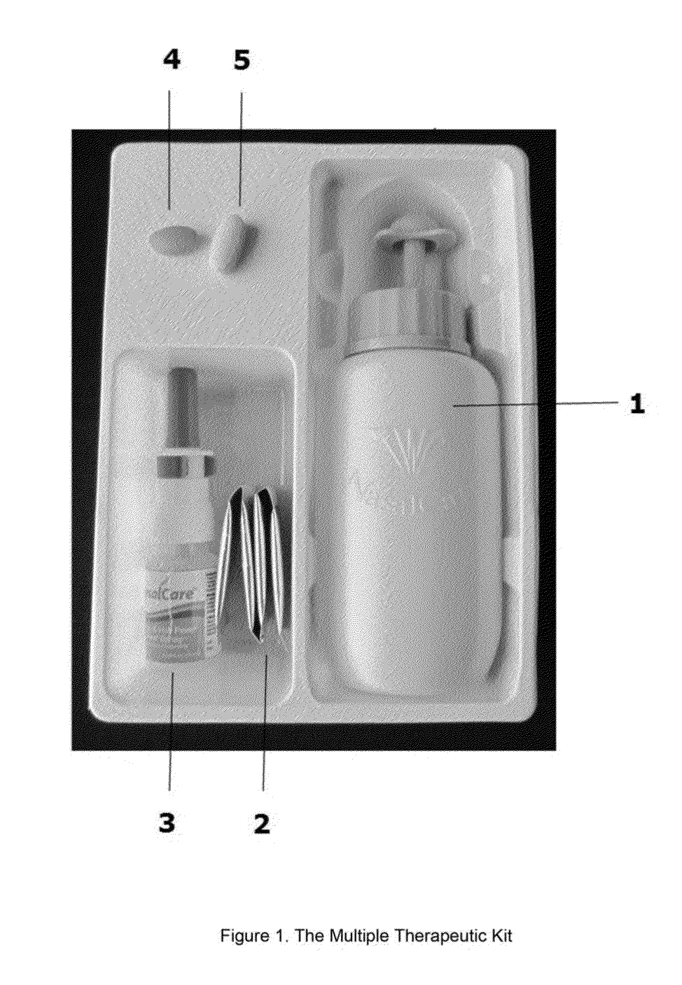

FIG. 1 is a photo of the Multiple Therapeutic Kit. It composes (1) a nasal irrigator; (2) the mix packet to be used for making the nasal cleanse solution; (3) a spray for delivering a therapeutic drug to nasal cavity, oral cavity, or any part of the body as appropriate; (4) a pharmaceutical preparation for a special therapeutic effect; (5) a pharmaceutical preparation for promoting self healing.

The combination of (1) the nasal irrigator and (2) the mix packet for making the nasal cleanse solution is used to physically cleanse out microbes, dusts, allergens, inflammatory factors, or excessive mucus from nasal cavities. The volume of the solution can vary from 10 to 300 ml. Preferably, it is between 120 to 240 ml for an effective nasal irrigation. The concentration of the irrigation solution can be isotonic or hypertonic. The solution is pH balanced with sodium citrate and citric acid, as well sodium bicarbonate.

The spay (3) can be nasal spray, oral spray, head spray, ear spray, or any body part spray as needed. The volume of the spray can vary from 0.5 to 100 ml. Preferably, the volume is between 30 to 60 ml.

The pharmaceutical preparation for a special therapeutic effect (4) is for anti-inflammation, anti-allergy, anti-autoimmune reaction, or anti-cancerous growth. The dosage can vary from 0.1 mg to 1.0 gram depending on the actual pharmaceutical ingredient used. The size of the pill or capsule can be any size of the typical drug. The pharmaceutical preparation for promoting self healing (5) is for enhancing immunity, regulating metabolism, promoting cellular or tissue growth or repair, or building a physiological potential to reduce the risk of developing the specific disorder. The dosage can vary from 0.1 mg to 1.0 gram depending on the actual pharmaceutical ingredient used. The size of the pill or capsule can be any size of the typical drug.

### DETAILED DESCRIPTION OF THE INVENTION AND THE NEW PROCESS OF USE

To overcome the lack-effectiveness of the singly use of either nasal irrigation or anti-inflammatory drug in treating a variety of diseases, the present invention provides a combinational therapy of eliminating microbes and/or other harmful substances from sinonasal cavities, as well as a pharmaceutical composition comprising the agent that has an activity of anti-inflammation, anti-allergy, anti-autoimmune reaction, or anti-cancerous growth, or has an activity for enhancing immunity, regulating metabolism, promoting cellular or tissue growth or repair, or building a physiological potential for self healing, and for reducing the risk of developing the specific disorder.

The use of the medical device in the current invention has been clinically proven to physically cleanse out these viruses, bacteria, fungi, allergens, dustings, or inflammatory factors. Particularly, its anti-viral effect is to meet the unmet need of treating viral infections. Many of these viral infections are lack of specific anti-viral drugs or vaccines for treatment or prevention. The physical elimination by nasal cleanse is cost-effective, and the procedure is safe and easy to perform.

The medical device of the present invention are further capable of eliminating certain inflammatory factors on the surface of the respiratory tract, enhancing the natural mucociliary clearance, promoting mucolytic activity, reducing airway resistance, easing inspiration and expiration, and increasing blood oxygenation of the lung.

The combinational use of a medical device and a pharmaceutical composition of the present invention are particularly suitable for the treatment or prevention of infections by bacteria selected from the group consisting of Streptococcus pneumoniae, Heamophilus influenzae, Staphylococcus aureus, or Staphylococcus epidermis.

The combinational use of a medical device and a pharmaceutical composition of the present invention are suitable for the treatment or prevention of the infection caused by fungus. The pharmaceutical compositions of the present invention are also suitable for the treatment or prevention of a mixed infection by virus, bacterium, and a fungus.

The pharmaceutical compositions are also homeopathic remedies which act by stimulating individuals to heal themselves and are selected based on the Law of Similars: “let likes be cured by likes.” The homeopathic formulation is capable of reducing the severity of a wide variety of ailments including infections, allergies, asthma, autoimmune diseases, and metabolic diseases. These homeopathic components may include, but not limited to: Aconite, Aconitum napellus, Adrenalinum, Allium Cepa, Ambrosia, Artemisiaefolia, Antimonium crudum, Antimonium tartaricum, Apis Mellifica, Aralia Racemosa, Argentum Metallicum, Arnica, Arsenicum, Arsenicum Album, Arum ax, Causticum, Arum Triphyllum, Arundo Mauritanica, Baptisia tinctoria, Baryta carbonica, Belladonna, Blatta Orientalis, Bryonia, Carbo vegetabilis, Causticum, Chamomilla, Colocynthis, Drosera, Dulcamara, Echinacea Angustifolia, Echinacea Purp., Eriodictyon Californicum, Eucalyptus, Eucalyptus Globulus, Eupatorium perfoliatum, Euphrasia, Euphrasia Officinalis, Ferrum phosphoricum, Gelsemium Sempervirens, Graphites, Grindelia Robusta, Hepar Sulphuris Calcareu, Histaminum, Hydrochloricum, Hydrastis Canadensis, Ignatia Amara, Influenzinum, Ipecacuanha, Kali Bichromicum, Kali sulphuricum, Lachesis, Lemna Minor, Lobelia Inflata, Lycopodium Clavatum, Mentha Piperita, Mercurius Corrosivus, Mercurius solubilis, Mercurius vivus, Naphthalinum, Natrum muriaticum, Natrum sulphuricum, Nicculum metallicum, Nux vomica, Penthorum, Phosphorus, Phytolacca, Pothos foetida, Pulsatilla, Pyrogenium, Quebracho, Rhus toxicodendron, Sabadilla, Sanguinaria Canadenis, Sarcolacticum Ac., Selenium Metallicum, Senega, Spongia tosta, Squilla Maritima, Sulphur, Thuja Occidentalis, Urtica Urens, Wyethia, Wyethia, Heleniodes, Zincum Gluconic.

The present invention further provides a method of treatment and/or prevention of respiratory disorders in a mammal by the combination of eliminating pathogenic factors and administering to the mammal an effective amount (e.g., therapeutically or prophylactically effective) of a pharmaceutical material readily available to the public, such as acetaminophen, ibuprofen, ketoprofen, diclofenac.

The pharmaceutically acceptable carriers described herein, for example, vehicles, adjuvants, excipients, or diluents, are well-known to those who are skilled in the art and are readily available to the public. It is preferred that the pharmaceutically acceptable carrier be one which is chemically inert to the active compounds and one which has no detrimental side effects or toxicity under the conditions of common use.

The choice of carrier will be determined in part by the particular active agent, as well as by the particular method used to administer the composition. Accordingly, there are a wide variety of suitable formulations of the pharmaceutical composition of the present invention. The following formulations for oral, intranasal, intrasinal, aerosol, subcutaneous, intravenous, intraarterial, intramuscular, interperitoneal, rectal, and vaginal administration are merely exemplary and are in no way limiting.

Formulations suitable for oral administration can consist of (a) solutions, such as an effective amount of the compound dissolved in diluents, such as water, saline, milk or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents; moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day if desired. In proper doses and with suitable administration of certain compounds, the present invention provides for a wide range of responses. The dosages range from about 0.001 to about 1000 mg/kg body weight of the mammal being treated per day. Preferred dosages range from about 0.01 to about 10 mg/kg body weight/day, and further preferred dosages range from about 0.01 to about 1 mg/kg body weight/day.

The present invention provides a delivery system to administer a liquid formulation directly into sinonasal cavities. Pharmaceutical preparations, such as a therapeutic agent, can be administered into nasal cavities.

The following examples further illustrate the present invention but, of course, should not be construed as in any way limiting its scope.

### Example 1

This example illustrates the efficacy of using the combinational therapeutic approach to treat a common cold. An adult male patient with the symptoms of the common cold, sore throat, sneeze, cough, and nasal congestion, performed nasal irrigation four times a day using an anti-backwash nasal irrigation system on the first day of the disease, roughly at 10 am, 2 pm, 6 pm and 10 pm. In between these nasal irrigation procedures, he used a homeopathic nasal spray to add the treatment efficacy. He also took Tylenol to control his symptoms following the label instruction. His symptoms were all disappeared the next day. This combinational treatment cured his common cold very fast.

### Example 2

This example illustrates the efficacy of using the combinational therapeutic approach to prevent common cold or influenza. Five healthy adults who had the cold or flu in the previous winter performed daily nasal irrigation with an anti-back wash nasal cleanse system each evening starting in middle of November 1 till middle of February next year. They also used a homeopathic nasal spray in each morning. During the entire period, no one developed common cold or flu. This combinational approach prevented the occurrence of common cold or flu.

### Example 3

This example illustrates the efficacy of using the combinational therapeutic approach to prevent asthma. An adult female patient with many years history of moderate asthma performed daily nasal irrigation each evening. In addition, she used a homeopathic nasal spray each morning. During the four week period, she did not have a single asthma flare up. Two weeks after she stopped performing these two procedures, she had an asthma attack. She then resumed to perform a daily nasal cleanse and did nasal spray for the rest of the winter. She did not have another asthma attack. This combinational approach prevented the occurrence of asthma for this patient.

### Example 4

This example illustrates the efficacy of using the combinational therapeutic approach to prevent the occurrence frequency of chronic obstructive pulmonary disease (COPD) symptoms. An old male patient with many years history of COPD performed daily nasal irrigation with an anti-back wash nasal cleanse system each evening. In addition, he used a homeopathic nasal spray each morning. During the four week period, he did not have a single occurrence of COPD symptoms. This combinational approach prevented the occurrence of COPD symptoms for this patient.

### Example 5

This example illustrates the efficacy of using the combinational therapeutic approach to treat influenza. An adult female patient with the flu symptoms, sneeze, cough, fever, muscle pain, lost appetite, performed nasal irrigation four times a day using an anti-backwash nasal irrigation system on the second day of the disease, at about 7 am, 11 am, 3 pm, and 8 pm. In between these nasal irrigation procedures, she used a homeopathic nasal spray to add the treatment efficacy. She also took Tylenol to control her symptoms three times a day. Her symptoms disappeared two days after she took the combinational therapy. This combinational treatment cured her flu very quickly.

### INDUSTRIAL APPLICABILITY

Through the easy and safe use of the combinational methods of this invention to cleanse out viruses or these triggers of asthma, the general population now has a simple, safe and inexpensive method that can be used to eliminate any known or unknown respiratory viruses, with added protection from using a homeopathic nasal spray to treat and prevent a human disease. For a medical professional, she/he can show to the patient how to use this simple and effective method to cure or prevent these common diseases. The significant advantage of using this new combinational system is that there will be no chance to develop multiple-drug resistance when a person to have viral infection and to take an antibiotics, which is a big public threat in light of many deaths caused by those super bugs. Hence, those drug's adverse effects and disturbance of immunity can be avoided. The medical community and the general population will greatly benefit from using the combinational method disclosed herein. The medical community now has a simple and reliable method to let the patient to recover fast from common cold, flu, and other respiratory tract disorders.
A publication entitled: Pharmaceutics and Pharmacy Practice, J.B. Lippincott Co., Philadelphia, Pa., Banker and Chalmers, eds., pages 1982, 238-250.
A publication entitled: The American Academy of Otolaryngology-Head and Neck Surgery (AO-HMS), http://www.entnet.org/healthinfo/sinus/sinus_questions.cfm, 2007.
A publication entitled: The diagnosis and incidence of allergic fungal sinusitis, Ponikau, J. U., D. A. Sherris, E. B. Kern, H. A. Hamburger, E. Frigas, T. A. Gaffey, and G. D. Roberts. 1999. Mayo Clin. Proc. 74:877-884.
A publication entitled: Pharmacy Practice, J.B. Lippincott Co., Philadelphia, Pa., Banker and Chalmers, eds., 1982, pages 238-250.
A publication entitled: ASHP Handbook on Injectable Drugs, 1986, Toissel, 4th ed pages 622-630.
A publication entitled: Microbiology of the maxillary and ethmoid sinuses in patients with chronic rhinosinusitis submitted to functional endoscopic sinus surgery. Nigro et al, Rev Bras Otorrinolaringol 2006, Vol. 72(2):217-222.
A publication entitled: Efficacy and mechanism of nasal irrigation with a hand pump against influenza and non-nfluenza viral upper respiratory tract infection. Ao et al. Journal of Infectious Diseases and Immunity 2011, Vol. 3(6), pp. 96-105.
A publication entitled: Virus-induced Airway Hyperresponsiveness and Asthma. Folkerts et al. Am J Respir Crit Care Med 1998, Vol 157. pp 1708-1720.
A publication entitled: Neutrophil degranulation and cell lysis is associated with clinical severity in virus-induced asthma. Wark et al. Eur Respir J 2002; 19: 68-75.
A publication entitled: An Evaluation of Echinacea angustifolia in Experimental Rhinovirus Infections. Turner et al. N Engl J Med 2005; 353:341-8.
A publication entitled: Asthma Exacerbations in Children Associated with Rhinovirus but not Human Metapneumovirus Infection. Rawlinson at al. The Journal of Infectious Diseases 2003; 187:1314-8.
A publication entitled: Effect of Interactions Between Lower Airway Bacterial and Rhinoviral Infection in Exacerbations of COPD. Wilkinson et al. CHEST 2006; 129:317-324.
A publication entitled: Exacerbations of Chronic Obstructive Pulmonary Disease. Wedzicha et al. RESPIRATORY CARE•DECEMBER 2003 VOL 48 NO 12, 1204-1215.

Reference:

| | | | |
|---|---|---|---|
| 1,248,891 | December, 1917 | Nichols | 604/289. |
| 1,487,252 | March, 1924 | Lore | 604/289. |
| 1,502,163 | July, 1924 | Sprague | 604/217. |
| 2,566,806 | September, 1951 | Miller | 128/173. |
| 2,582,529 | January, 1952 | Curry | 128/200. |
| 2,693,800 | November, 1954 | Caldwell | 604/289. |
| 3,847,145 | November, 1974 | Grossan | 601/160. |
| 4,029,095 | June, 1977 | Pena | 128/250. |
| 4,403,611 | September, 1983 | Babbitt et al. | 604/118. |
| 4,980,163 | December, 1990 | Blackburn et al. | 434/94. |
| 5,116,311 | May, 1992 | Lofstedt | 604/54. |
| 5,183,467 | February, 1993 | Mouney | 604/140. |
| 5,314,690 | May, 1994 | Patterson et al. | 424/114. |
| 5,505,193 | April, 1996 | Ballini et al. | 128/200. |
| 5,752,510 | May, 1998 | Goldstein | 128/200. |
| 5,794,619 | August, 1998 | Edelman et al. | 128/200. |
| 6,238,377 | May, 2001 | Liu | 604/289. |
| 6,736,792 | May, 2004 | Liu | 598/522. |

### OTHER REFERENCES

A publication entitled: “Parainfluenza viral infections in children: Correlation of shedding with clinical manifestations” by Hall et al. in The Journal of Pediatrics, 1982, Vol. 91, No. 2, pp. 194-198.
A publication entitled “Clinically Useful Method for the Isolation of Respiratory Syncytial Virus” by Hall et al. in The Journal of Infectious Diseases, 1975, Vol. 131, No. 1, pp. 1-5.
A publication entitled: “Quantitative Shedding Patterns of Respiratory Syncytial Virus in Infants” by Hall et al. in The Journal of Infectious Diseases, 1975, Vol. 132, No. 2, August, pp. 151-156.
A publication entitled: New advances in aerosolised drug delivery: vibrating membrane nebuliser technology by Lass et al. in Expert Opinion on Drug Delivery 2006, Vol. 3, No. 5, Pages 693-702.

What is claimed is:

1. A kit providing multiple unmet therapeutic effects comprising:
an anti-backwash sinonasal cleanse system for eliminating pathogenic materials from sinonasal cavities and nasopharynx for preventing and treating a disease of a mammal;
a pharmaceutical preparation with a strong anti-inflammation activity for treating a disease of a mammal;
a homeopathic formulation capable of preventing and treating a disease of a mammal.

2. The anti-backwash sinonasal cleanse system of claim 1, wherein the said at least can effectively cleanse out pathogenic microbes causing respiratory infections.

3. The anti-backwash sinonasal cleanse system of claim 1, wherein the said at least can effectively cleanse out common allergens causing allergic diseases.

4. The anti-backwash sinonasal cleanse system of claim 1, wherein the said at least can effectively cleanse out the excessive mucus in sinonasal cavities.

5. The pharmaceutical preparation with a strong anti-inflammation activity of claim 1, wherein the said at least has one of these commonly used anti-inflammatory agents, such as acetaminophen, ibuprofen, ketoprofen, diclofenac.

6. The homeopathic formulation of claim 1, wherein the said at least has any three of the homeopathic ingredients: Aconite, Aconitum napellus, Adrenalinum, Allium Cepa, Ambrosia, Artemisiaefolia, Antimonium crudum, Antimonium tartaricum, Apis Mellifica, Aralia Racemosa, Argentum Metallicum, Arnica, Arsenicum, Arsenicum Album, Arum ax, Causticum, Arum Triphyllum, Arundo Mauritanica, Baptisia tinctoria, Baryta carbonica, Belladonna, Blatta Orientalis, Bryonia, Carbo vegetabilis, Causticum, Chamomilla, Colocynthis, Drosera, Dulcamara, Echinacea Angustifolia, Echinacea Purp., Eriodictyon Californicum, Eucalyptus, Eucalyptus Globulus, Eupatorium perfoliatum, Euphrasia, Euphrasia Officinalis, Ferrum phosphoricum, Gelsemium Sempervirens, Graphites, Grindelia Robusta, Hepar Sulphuris Calcareu, Histaminum, Hydrochloricum, Hydrastis Canadensis, Ignatia Amara, Influenzinum, Ipecacuanha, Kali Bichromicum, Kali sulphuricum, Lachesis, Lemna Minor, Lobelia Inflata, Lycopodium Clavatum, Mentha Piperita, Mercurius Corrosivus, Mercurius solubilis, Mercurius vivus, Naphthalinum, Natrum muriaticum, Natrum sulphuricum, Nicculum metallicum, Nux vomica, Penthorum, Phosphorus, Phytolacca, Pothos foetida, Pulsatilla, Pyrogenium, Quebracho, Rhus toxicodendron, Sabadilla, Sanguinaria Canadenis, Sarcolacticum Ac., Selenium Metallicum, Senega, Spongia tosta, Squilla Maritima, Sulphur, Thuja Occidentalis, Urtica Urens, Wyethia, Wyethia, Heleniodes, Zincum Gluconic.

7. A process of using the kit containing an anti-backwash sinonasal cleanse system, a pharmaceutical preparation with a strong anti-inflammation activity, a homeopathic formulation to prevent and treat common respiratory tract diseases of a mammal comprising:
performing a daily sinonasal cleanse at least one time a day for preventing a disease;
performing a daily sinonasal cleanse at least two times a day for treating a disease;
administering an anti-inflammation pharmaceutical preparation for treating a disease by following the label instruction;
administering a homeopathic formulation at least one time a day for preventing a disease;
administering a homeopathic formulation at least two times a day for treating a disease.

8. The common respiratory tract diseases of claim 7, wherein the said is a common cold.

9. The common respiratory tract diseases of claim 7, wherein the said is influenza.

10. The common respiratory tract diseases of claim 7, wherein the said is asthma.

11. The common respiratory tract diseases of claim 7, wherein the said is sinusitis.

12. The common respiratory tract diseases of claim 7, wherein the said is nasal allergy.

13. The common respiratory tract diseases of claim 7, wherein the said is chronic obstructive pulmonary disease.

14. A method of using the kit containing an anti-backwash sinonasal cleanse system, a pharmaceutical preparation with a strong anti-inflammation activity, a homeopathic formulation to prevent and treat common respiratory tract diseases of a mammal comprising:
using the anti-backwash sinonasal cleanse system to eliminate these pathogenic substances from sinonasal cavities;
using a pharmaceutical preparation to suppress inflammation;
using a homeopathic formulation to build up resistance to a disease.

15. The common respiratory tract diseases in accordance with claim 14, wherein the said is an infectious disease.

16. The common respiratory tract diseases in accordance with claim 14, wherein the said is an allergic disease.

17. The common respiratory tract diseases in accordance with claim 14, wherein the said is chronic obstructive pulmonary disease.

18. The common respiratory tract diseases in accordance with claim 14, wherein the said is asthma.

19. The common respiratory tract diseases in accordance with claim 14, wherein the said is a sinusitis.

20. The common respiratory tract diseases in accordance with claim 14, wherein the said is a post nasal drip.
